(19) United States
(12) Patent Application Publication
(10) Pub. No.: US 2002/0107499 A1
(43) Pub. Date: Aug. 8, 2002

(54) Method and apparatus for facilitating fluid retrieval from medicinal receptacles

(76) Inventor: Steven Ross Funk, Apple Valley, MN (US)

Correspondence Address: Steven R. Funk, 8111 Upper 129th Ct., Apple Valley, MN, 55124, US

(21) Appl. No.: 09/779,206

(22) Filed: Feb. 8, 2001

(51) Int. Cl. A61M 5/00; A61B 19/00

(52) U.S. Cl. 604/403; 206/828

(57) ABSTRACT

A method and apparatus for maximizing fluid retrieval from a fluid container when using a fluid retrieval device. A medical container is provided which includes a container body to hold a volume of medicinal fluid, and a container nozzle coupled to the container body. The nozzle has an opening to permit passage of a fluid retrieval device into the container body. The container includes a fluid pooling structure that guides the volume of medicinal fluid towards a target pooling region in response to a gravitational force, thereby optimizing the fluid retrieval therefrom.

### FIELD OF THE INVENTION

This invention relates in general to fluid containers, and more particularly to a method and apparatus for facilitating complete retrieval of medicinal fluids from a medicinal container.

### BACKGROUND OF THE INVENTION

Children who become ill or otherwise require medication are often medicated orally. Babies and toddlers are not capable of receiving medicines in the form of a pill, and are generally subjected to medicinal products in a liquid form that can be taken orally. Because most medicinal remedies are administered to babies and toddlers in this fashion, and because children of these ages often require medications such as antibiotics, aspirin, cough and cold medicine, etc., parents and other caretakers of the children are regularly having to administer these fluid medications. Along with children, the elderly, infirm, physically disabled, etc. are also candidates for drug administration via oral ingestion.

One problem with administering such medicines is that babies and small children do not take liquid medicines from a spoon very well. Babies cannot form their mouth around a spoon, and toddlers, even if capable of receiving medications via spoon, are often uncooperative. These problems generally lead to spillage, or otherwise result in an inadequate dosage of administered medicine.

One solution to this problem is to use a syringe to draw fluid medicine from a medicine bottle, and administer the medicine with the syringe. The syringe typically has a blunt end that can be safely inserted into the child's mouth. Once the syringe is filled, the syringe plunger is depressed to expel the fluid into the child's mouth. This allows the medicine to be more accurately released into the child's mouth, as a result of the ability to position the medicinal release point in the child's mouth and direct the fluid into the mouth. Further, there is a greatly enhanced ability to control the speed and quantity of fluid release.

However, when the fluid level in the medicine container decreases, it becomes increasingly difficult to draw fluid into the syringe. This can be a result of the syringe having insufficient length to reach the bottom of the container. However, even where the syringe is of sufficient length to reach the bottom of the container, the fact that the remaining fluid is distributed over the entire bottom area of the container makes it difficult to draw the remaining portion of the medicine into the syringe. For example, as the syringe plunger is pulled to draw in fluid, it creates suction at the fluid entry point. The suction that is generated may pull some fluid into the syringe, but also pulls air into the syringe when the fluid level has decreased to a certain point. In order to fill the syringe, it may need to be iteratively tipped upside-down to release any captured air, and an attempt to draw in additional fluid must then be made. Further, in order to draw in the last portions of the fluid, the end of the syringe must be positioned proximate the bottom structure of the container, making fluid suction difficult as a result of the syringe end being positioned against the bottom of the container.

Alternatively, in an attempt to draw in any remaining fluid, the container can be tipped to a side to try to pool the medicine between the edge and the bottom of the container. This also has disadvantages. For example, if the diameter of the syringe is close to the diameter of the container opening, there is relatively little, if any, ability to insert the syringe at an angle with respect to the opening of the medicine container, since the rigidity of the medicine container causes the syringe to be positioned in line with the container channel opening. Even where the diameter of the syringe is less than the diameter of the container opening, there is only a limited angle to which the syringe can be inserted into the container.

The medicine could be poured from the medicinal container into another container, such as a small bowl or small medicine cup. In this way, the medicinal syringe is uninhibited by the medicinal container itself, and can draw in the medicine. However, it is very inconvenient to have to pour the medicine into another container, especially where any excess that was poured must be poured back into the medicinal container or discarded. If poured back into the medicine container, this creates yet another inconvenience, and causes waste of the medicine as portions of the medicine cling to the walls of the bowl or medicine cup. Further, it may be difficult to pour the unused medicine back into the container, and may require a funnel or other means of pouring the fluid into the small container opening. The alternative is to discard any remaining, unused portion of the medicine that was poured from the container into the bowl or cup, but it is clearly an undesirable action to waste costly medicines, particularly where the medicine is a prescription drug and only a limited amount is prescribed to the patient.

It can be seen that there is a need for a method and apparatus that facilitates substantially complete removal of the medicinal fluids from a medicine container when used in connection with a retrieval device, such as a syringe or other suction device. It would be desirable to avoid the aforementioned and other problems associated with conventional containers and techniques to minimize waste and provide ease of use. The present invention provides a solution to the aforementioned and other problems of the prior art, and offers additional advantages over current medicinal delivery devices and techniques.

### SUMMARY OF THE INVENTION

To overcome the limitations in the prior art described above, and to overcome other limitations that will become apparent upon reading and understanding the present specification, the present invention discloses an apparatus and method for maximizing fluid retrieval from a fluid container when using a fluid retrieval device.

A medical container is provided in accordance with one embodiment of the invention. The container includes a container body to hold a volume of medicinal fluid, and a container nozzle coupled to the container body. The nozzle has an opening to permit passage of a fluid retrieval device into the container body. The container further includes a fluid pooling structure that guides the volume of medicinal fluid towards a target pooling region in response to a gravitational force.

Various more specific embodiments of the invention set forth particular fluid pooling structures, which may take on a variety of forms, including a conical form, an inclined planar form, a V-shape, and the like. Other more particular embodiments include a grating at the target pooling region to facilitating fluid retrieval between a fluid receiving end of the fluid retrieval device and the target pooling region.

Still other specific embodiments include a coupling mechanism on the container nozzle and on the fluid retrieval device to allow the fluid retrieval device to be coupled to the container. This may be beneficial as a capping technique for storage and transport of the container, and also to provide the proper positioning of a fluid receiving end of the fluid retrieval device proximate the target pooling region.

In accordance with another embodiment of the invention, a medicinal fluid dispensing system is provided. The system includes a medicinal container that has a container body to hold the medicinal fluid. A container nozzle is coupled to the container body and has an opening therein. A fluid pooling structure is arranged to direct the medicinal fluid to a target pooling region in response to gravity. The system also includes a fluid retrieval device configured to pass through the opening in the nozzle, where the container nozzle is configured to direct the fluid retrieval device towards the target pooling region to retrieve the medicinal fluid pooled at the target pooling region. In more specific embodiments, the fluid retrieval device includes a syringe, a dropper, or other device capable of drawing in the medicinal fluid.

In accordance with another aspect of the invention, a method is provided for maximizing fluid extraction from a container. Directional access is provided in the container for passage of a fluid retrieval device therethrough. The fluid converges to a fluid collection area having decreased volumetric dimensions as compared to the container dimensions. The longitudinal axis of the container access is substantially aligned with the fluid collection area. A fluid retrieval device inserted into the container via the directional access is directed to the fluid collection area, as governed by the container access and aligned fluid collection area, thereby maximizing fluid extraction quantity from the container.

In still another embodiment of the invention, another method for maximizing fluid extraction from a medicinal container is provided. A fluid retrieval device is inserted into a medicinal container nozzle at an angle defined by the medicinal container nozzle. The fluid retrieval device is directed along its longitudinal axis until reaching an inner wall of the container. An angle of the medicinal container itself is adjusted to pool the fluid proximate an end of the fluid retrieval device. The fluid is retrieved by suctioning the fluid proximate the pooled fluid.

These and various other advantages and features of novelty which characterize the invention are pointed out with particularity in the claims annexed hereto and form a part hereof. However, for a better understanding of the invention, its advantages, and the objects obtained by its use, reference should be made to the drawings which form a further part hereof, and to accompanying descriptive matter, in which there are illustrated and described specific examples of an apparatus in accordance with the invention.

### BRIEF DESCRIPTION OF THE DRAWINGS

FIG. 1 generally illustrates one embodiment of a fluid container in accordance with the present invention;

FIG. 2 illustrates an embodiment of the invention wherein a fluid container and fluid retrieval device in accordance with the invention is inserted into the medicinal container;

FIG. 3 is an embodiment of the invention wherein the fluid retrieval device is a dropper device;

FIG. 4 illustrates an embodiment of the invention where the fluid retrieval device is a syringe-type device including a threaded structure to secure the syringe to the medicinal bottle;

FIG. 5 illustrates another embodiment of a bottom portion of a medicinal container in accordance with the present invention;

FIGS. 6 and 7 illustrate embodiment of fluid pooling structures in accordance with the present invention;

FIGS. 8 and 10 illustrate the meeting of a fluid retrieval device with the target pooling region, where a shallow fluid grate is provided to facilitate retrieval of the fluids when the tip of the fluid retrieval device is in juxtaposition with the target pooling region;

FIG. 9 illustrates the dilemma posed where no fluid grate is provided to facilitate retrieval of the fluids when the tip of the fluid retrieval device is in juxtaposition with the target pooling region;

FIGS. 11-15 illustrate various example embodiments of grates that can be used in connection with the target pooling regions of the present invention;

FIG. 16 illustrates one embodiment where no grate is necessary at the target pooling region;

FIG. 17 illustrates an embodiment of a fluid retrieval device which works in connection with a pooling structure, while also serving as a removable cover to the container;

FIGS. 18 and 19 illustrate various embodiments of a dropper device in accordance with the present invention;

FIG. 20 illustrates an embodiment of the invention wherein a fluid container and fluid retrieval device in accordance with the invention is inserted into the medicinal container at a predetermined angle to appropriately reach the target pooling region;

FIG. 21 is a flow diagram of a method for retrieving fluid from a container having an angled nozzle; and

FIG. 22 is a flow diagram generally illustrating a method for maximizing the removal of fluids from a medicinal fluid container in accordance with the present invention.

### DETAILED DESCRIPTION OF THE INVENTION

In the following description of the exemplary embodiment, reference is made to the accompanying drawings which form a part hereof, and in which is shown by way of illustration specific embodiments in which the invention may be practiced. It is to be understood that other embodiments may be utilized, as structural and operational changes may be made without departing from the scope of the present invention.

The present invention is generally directed to medicinal container that facilitates fluid retrieval therefrom, particularly when used in connection with a fluid retrieval device capable of drawing in the medicinal fluid from the medicinal container. The medicinal bottle or container includes an opening from which a fluid retrieval device may be inserted, and further includes a fluid pooling structure substantially aligned with the container opening such that the fluid retrieval rod is directed through the opening substantially to the fluid pooling structure. In this manner, fluid may be withdrawn from medicinal containers with relative ease through the use of a fluid retrieval device/rod.

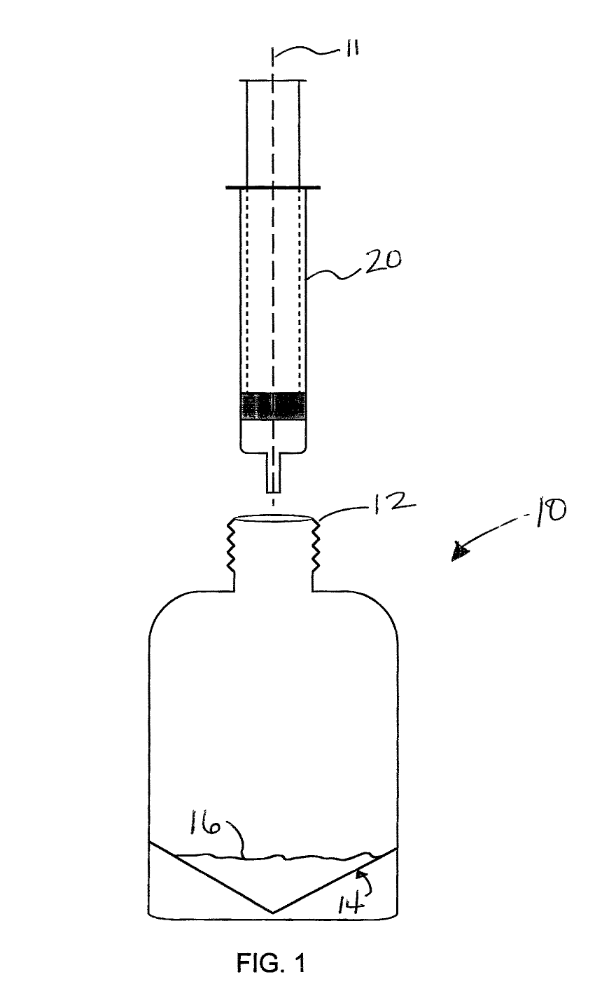

FIG. 1 generally illustrates one embodiment of the present invention. As shown in FIG. 1, a container 10 is provided for the storage and transport of fluids, such as liquid medicines. The container 10 may be any desired shape, and in the illustrated embodiment is generally the shape of a cylinder with an opening or nozzle 12. The container is constructed of a substantially watertight material that prevents absorption or leakage of the fluid contained therein. The medicinal container 10 includes a fluid pooling structure 14 that facilitates the pooling of the medicinal fluid proximate the structure 14 when placed or held in a position where gravitational force tends to direct the fluid towards the structure 14. A fluid retrieval device 20, 0which in the illustrated embodiment is a blunt-tip syringe, may enter the medicinal container 10 through the nozzle 12 to reach the fluid 16. When the fluid is at a high level in the container 10, the precise depth at which the fluid retrieval device is inserted into the container is of relatively little significance. However, when the fluid 16 level is at a low level in the container 10, traditional medicinal bottles distributed the little remaining fluid over a large service area at the relatively flat bottom of the medicinal container. Tipping such a traditional medicinal bottle to its side to allow a syringe to draw fluid from a point between a side wall and a bottom wall is typically ineffective, due to the physical dimensions of the nozzle 12 that directs the fluid retrieval device 20 such that it is unable to reach a point between the side wall and the bottom wall of the medicinal container. This is largely due to the relative rigidity of the fluid retrieval device 20. In the present invention, the fluid pooling structure 14 is designed such that the apex of a concave or conical structure, or more generally the center of the fluid pooling structure 14 depending on its shape, is substantially in line with a longitudinal axis 11 substantially through the center of the nozzle 12.

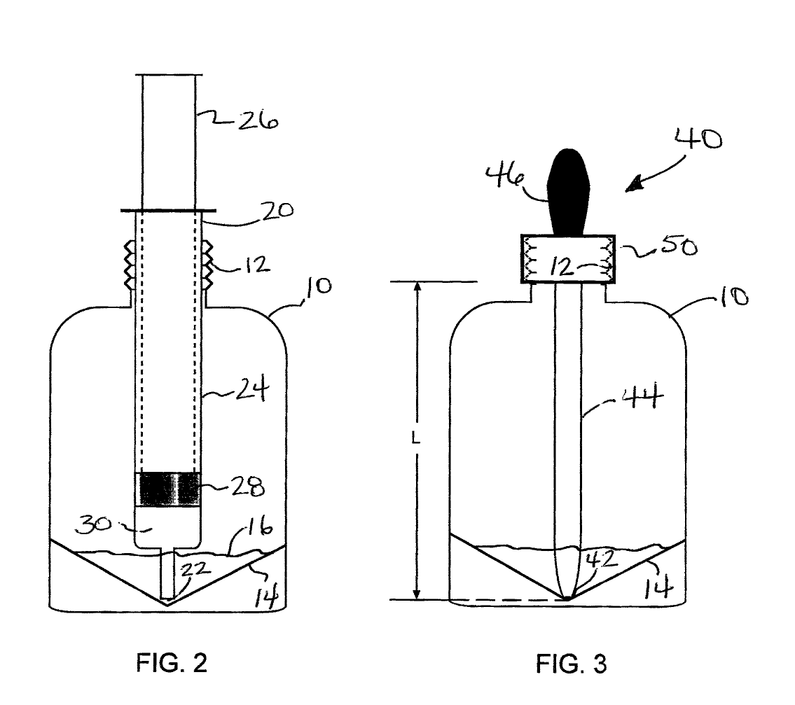

Referring now to FIG. 2, the medicinal container 10 and fluid retrieval device 20 shown in connection with FIG. 1 are shown in FIG. 2, with the fluid retrieval device 20 illustrated in its inserted position into the medicinal container 10. In this position, and due to the alignment of the longitudinal axis of the nozzle 12 with the center of the fluid pooling structure 14, the open tip 22 of the fluid retrieval device 20 is positioned such that the amount of fluid that may be drawn from the medicinal container 10 is maximized. The fluid retrieval device 20 includes an outer shell 24, inner plunger 26 having a rubber (or other elastomeric substance/material) ring 28 that snugly conforms within the inner diameter of the outer shell 24 sufficiently to create a vacuum when the inner plunger 26 is drawn out of the outer shell 24. Creation of this vacuum effect causes the fluid 16 to be drawn into the fluid retrieval device 20 via the open tip 22 into the vacuum chamber 30 created between the open tip 22 and the substantially air tight ring 28. As can be seen from FIG. 2, substantially all the fluid in the medicinal container 10 may be removed using a fluid retrieval device 20 where the longitudinal axis of the medicinal bottle nozzle 12 is directed in a line towards the pooling point or trough created by the pooling structure 14.

FIG. 3 is another embodiment of the invention wherein the fluid retrieval device is a dropper device 40. Such dropper devices are, of themselves, known in the art. In connection with the present invention, the dropper device 40 includes an open end tip 42, a substantially longitudinal body 44, and a bulb 46. Squeezing or otherwise depressing the bulb 46 forces the air inside the bulb 46 out of the dropper 40 through the open end tip 42. Releasing the bulb 46 then creates a vacuum within the dropper 40 to draw the fluids via the open end tip 42 into the vacuum chamber which is at least partially comprised of the longitudinal body 44. Insertion of a dropper 40 into a medicinal container 10 having a fluid pooling structure 14 in accordance with the present invention allows substantially all of the fluid stored in the container 10 to be withdrawn when the dropper has a length sufficient to reach the bottom of the fluid pooling structure 14.

It should also be recognized that other fluid retrieval devices other than a syringe-type device or dropper-type device may be used. For example, other devices using electronic suction mechanisms rather than manual suction mechanisms may be used. It is known to use motors to create vacuum effects, which can in turn be used to create the requisite suction to draw out the medicinal fluid from the medicinal container.

Optionally, the dropper 40 may include a threaded structure 50 positioned at an appropriate point on the dropper 40 such that the longitudinal body 44 fits within the medicinal container 10 when the threaded structure 50 is threaded onto a threaded nozzle 12. Other fastening mechanisms other than threads may also be used to attach the dropper 40 to the medicinal body 10 in accordance with the present invention. In any event, a fastening structure such as the threaded structure 50 allows the dropper 40 to also act as a cover or cap for the medicinal bottle 10 during periods of storage and transport.

In one particularly beneficial embodiment of the invention, the longitudinal body 44 has a length “L” such that when the threaded structure 50 is threaded onto the nozzle 12, the open end tip 42 is positioned proximate a pooling point or trough of the pooling structure 14. This pooling point or trough is a physical structure of the pooling structure 14 to which the fluid in the medicinal container is ultimately directed by gravity. In other words, as the fluid level decreases in the container 10, the remaining fluid in the container is directed to the pooling point, trough, or other pooling structure, examples of which are described in greater detail below. In this embodiment, the structure 50 serves as a cover, and when medicine is to be drawn, the bulb 46 can be squeezed before the “cover” is even removed, and when removed, the dropper 40 will contain a medicine dose.

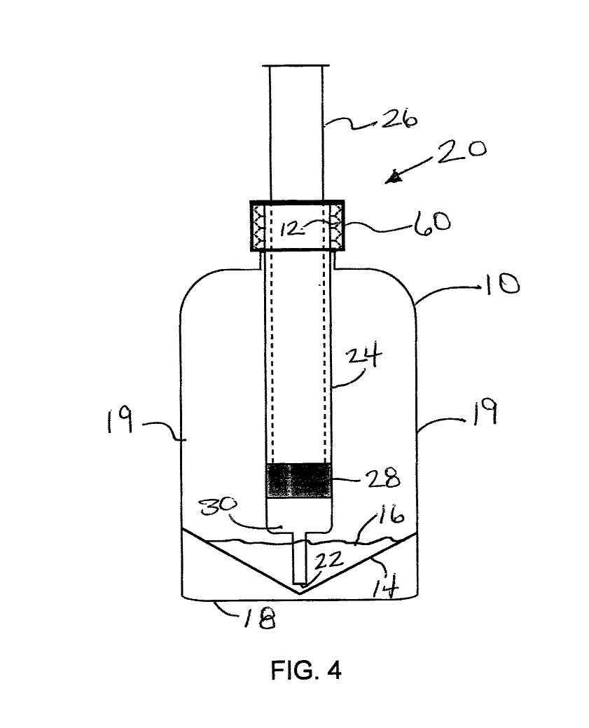

FIG. 4 illustrates another embodiment of the invention where the fluid retrieval device 20 is a syringe-type device as shown in FIGS. 1 and 2, but also includes a threaded structure 60 to secure the syringe to the medicinal bottle 10 during periods of non-use (e.g., storage, transport, etc.). Similar to the operation described in FIG. 3 in connection with the dropper 40, the syringe 20 shown in FIG. 4 can be threaded or otherwise connected to the medicinal bottle 10 via the structure 60 which is attached to the outer shell 24 of the syringe 20. The threaded structure 60 can then be threaded onto the threaded nozzle 12. Other fastening mechanisms other than threads can be used as well, as long as the structure 60 cooperates with the nozzle 12 to removably secure the structure 60 to the nozzle 12. As discussed previously, no securing mechanism 60 is necessary for purposes of the present invention, and the structure 60 represents one particular embodiment of the invention.

As shown in the examples of FIGS. 1-4, the medicinal bottle includes a pooling structure 14. In the examples of FIGS. 1-4, the pooling structure 14 is contained within the medicinal container 10. For example, the medicinal container 10 may include a bottom portion 18 as shown in FIG. 4. This bottom portion 18 and side walls 19 may, in this embodiment, form the exterior surface of the medicinal container 10. In such an embodiment, the pooling structure 14 is implemented within the container 10, such as by implementing a cone, trough, or other pooling structure 14 which defines the actual fluid boundary. The container bottom 18 in this example is thus a structural bottom to serve another purpose, such as to facilitate a stable base when the container 10 is set down on a surface.

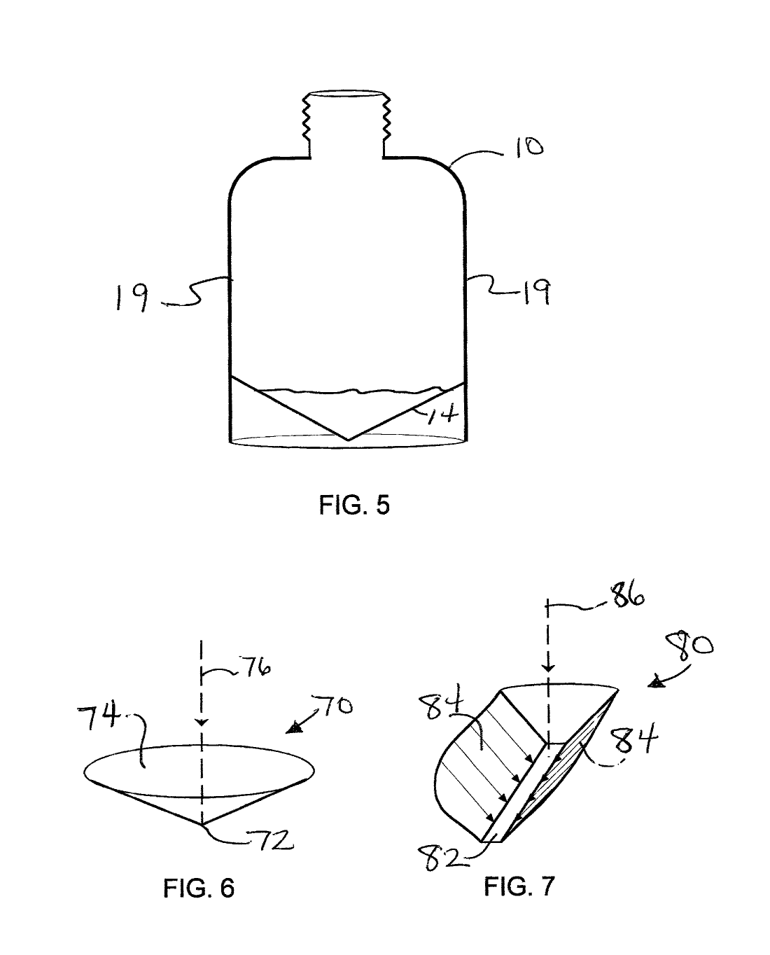

FIG. 5 illustrates another embodiment of a bottom portion of a medicinal container 10 in accordance with the present invention. In this embodiment, the cylindrical external portion of the container 10 includes only the side walls 19 and includes no container bottom 18 as was described in connection with FIG. 4. In the example of FIG. 5, the pooling structure 14 comprises the fluid boundary, and the circumference of the cylindrical ring forming the container side wall 19 provides the base to set the container 10 on a flat surface. Thus, in this embodiment, the pooling structure 14 also forms the bottom surface of the medicinal container 10, as compared to a pooling structure 14 that is essentially a “false bottom” in addition to a bottom surface 18 as was illustrated in FIG. 4.

FIG. 6 illustrates one embodiment of a fluid pooling structure in accordance with the present invention. In this embodiment, the pooling structure is represented by a substantially conical structure 70. The conical structure 70 includes a pooling point 72 and the fluid directing surface 74. The fluid directing surface 74 need not be uniform, but rather need only be such that it sufficiently tends to direct the fluid towards the pooling point 72 via gravity. When positioned such that gravity will direct fluid in a medicinal container 10 along the fluid directing surface 74 generally towards the pooling point 72, a fluid retrieval device generally directed towards the pooling point 72 substantially along an axis represented by dashed lines 76 will allow the fluid retrieval device to withdraw substantially all of the fluid from the medicinal container that is directed to the pooling point 72. It should be noted that the pooling “point” 72 need not be an actual cone point, but rather may be represented by a small planar surface, such that the conical structure 70 more closely resembles a cone with the point sliced off. In other words, the “point” 72 need not be well defined to come to a precise point, but rather may be a small planar platform to which the fluid directing surface 74 directs the fluid.

FIG. 7 illustrates another embodiment of a fluid pooling structure in accordance with the present invention. In this example embodiment, the pooling structure is represented by a V-shape or “trough” structure 80. In this embodiment, the area at which pooling occurs is not directed to a physical point, but rather towards a small physical line or trough 82. The trough 82 includes one or more fluid directing surface is 84, such that gravity will direct fluid in a medicinal container 10 along the fluid directing surface(s) 84 generally towards the trough 82. Again, the fluid directing surface(s) 84 need not be uniform or symmetric, but rather need only be arranged such that they sufficiently tend to direct the fluid towards the pooling trough 82 by way of gravity. In this embodiment, a fluid retrieval device inserted into the medicinal container 10 is generally directed along a path represented by dashed line 86 towards the trough 82, but is allowed some play in the exact position along trough 82. The trough 82 itself need not necessarily be planar as depicted in the example of FIG. 7, but rather the fluid directing surfaces 84 may simply meet to form a V-shape that forms the trough 82. Further, the trough may be a concave structure or other shape, as long as the fluid tends to be directed thereto by the fluid directing surfaces(s) 84.

The examples of FIGS. 6 and 7 are exemplary embodiments of the invention, and the invention is not, nor is intended to be, limited thereto. For example, other structures directing the fluid to a smaller point than that created by the bottom of a traditional container are contemplated within the scope and spirit of the present invention. For example, a partially conical surface may be used, leading to a longitudinally cylindrical structure in which the end or tip of a fluid retrieval device may enter to withdraw fluid. Other physical shapes that use gravity to direct the fluid to an area substantially in line with a longitudinal axis through the nozzle of a container are within the scope and spirit of the present invention.

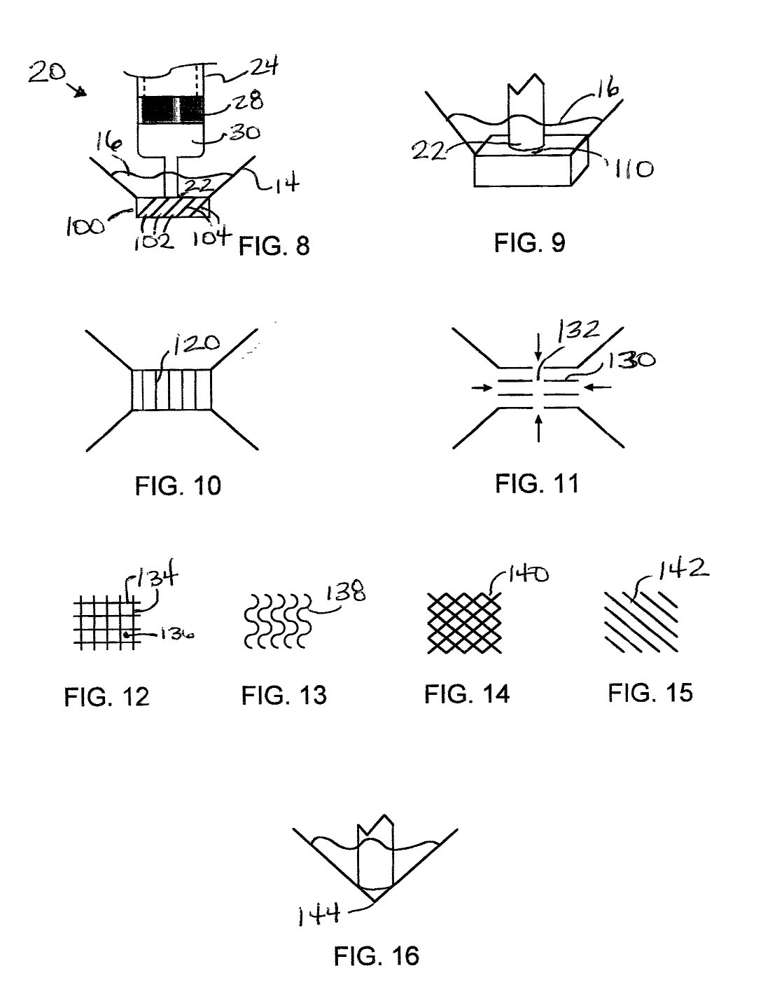

Fluid retrieval devices, such as the syringe 20 shown in FIG. 8, may include an open blunt tip 22 in which the fluid 16 may be drawn into the vacuum chamber 30. The pooling structure 14 directs the fluid 16 to a relatively small area in order to allow substantially all of the fluid to be drawn into the fluid retrieval device 20. In one embodiment of the invention, a shallow fluid grate 100 is provided at the pooling point or trough associated with the pooling structure 14. In this embodiment, the grate 100 prevents the tip 22 from pressing flat against a flat surface which could make it difficult for the fluid 16 to be drawn into the vacuum chamber 30. For example, if a plastic, flat end syringe were pushed against a flat surface containing fluid as shown in FIG. 9, the tip 22 and a bottom portion 110 essentially form a seal, making it difficult to draw the fluid 16 in through the tip. The grate 100 prevents this problem from occurring. Referring again to FIG. 8, fluid may enter fluid vias 102 of the shallow fluid grate 100. The grate segments 104 provide the platform on which the tip 22 will be positioned, but a small amount of fluid and/or air can enter the vias 102. In this manner, when fluid is withdrawn from the container 10, the tip 22 will not form a seal with a flat bottom surface of the pooling structure 14. This embodiment assumes that the distance between grate segments 104 are narrower than the opening of the tip 22. In this embodiment, substantially all of the fluid is retrieved from the container, although a insignificantly small amount may be unretrievable from the vias 102.

FIG. 10 illustrates a top view of a grate embodiment represented in FIG. 8. This embodiment is represented as an inverted trapezoid which directs fluid to a small grate 120. However, as described earlier, the directing surfaces may be conical and direct fluid to a point, small platform, or other shaped surface. The particular shape is not of particular relevance to the invention. For purposes of illustration, FIGS. 8 and 10-15 are depicted as rectangular, but again, could be any shape.

FIGS. 11-15 illustrate additional examples of grates, such as those described in connection with FIG. 8. The grate of FIG. 11 includes walls 130 and gaps 132 to allow fluid to enter the grate easily. The grate of FIG. 12 includes segments 134 at substantially right angles, forming substantially rectangular or square vias 136. The grates of FIGS. 13, 14 and 15 include wavy segments 138, non-perpendicular cross-hatching 140, and substantially parallel segments 142 respectively. These grates are illustrated for purposes of example only, and the invention is not limited thereto. Any grating structure or pattern allowing fluid to enter the grate, while keeping a fluid retaining device substantially atop the grate, may be implemented in accordance with the invention. Even random segments could be used, as long as fluid can enter the vias resulting from the segments, and the tip 22 is held substantially atop the grate structure. Further, the pooling structure may also be made so that no grate is necessary, such as a point or trough that is distinct enough such that a tip 22 of a fluid retrieval device cannot form a suction seal due to the point or vertex 144, as depicted in FIG. 16. The invention also contemplates pooling structures without any grating, or without any prevention from forming a suction seal between the fluid retrieval device and the bottom of the pooling structure.

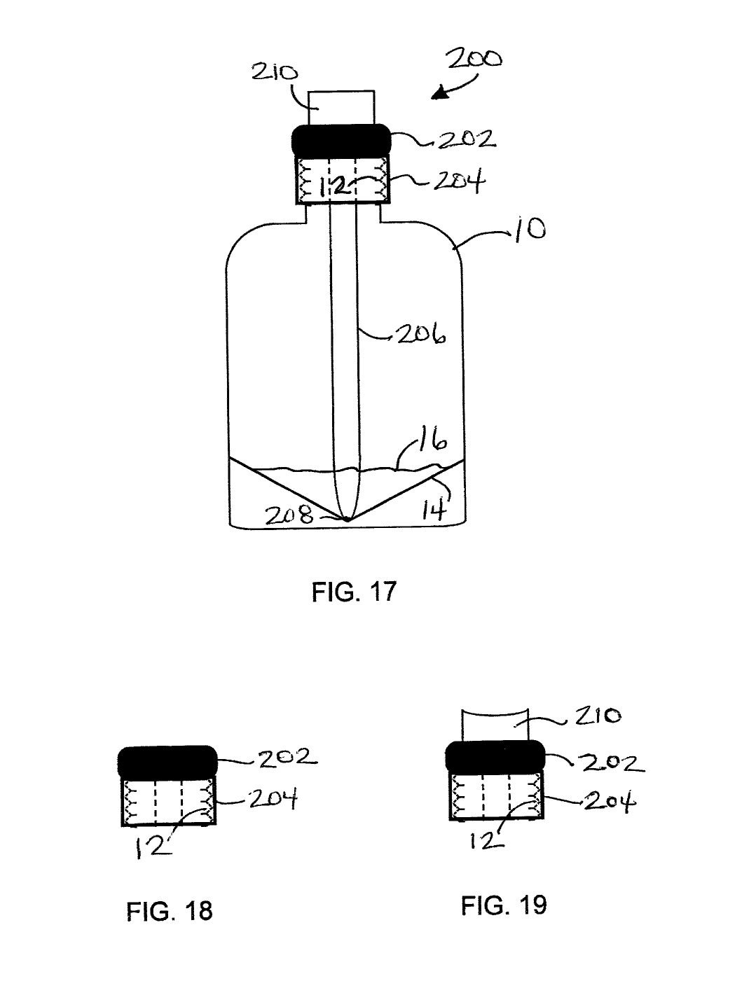

The fluid retrieval devices may be associated with the medicinal container such that it serves as a removable cover to the medicinal bottle. Such examples were previously described in connection with FIGS. 3 and 4. FIG. 17 illustrates another embodiment of a fluid retrieval device which works in connection with a pooling structure 14, while also serving as a removable cover. In the example embodiment of FIG. 17, a dropper 200 serves as a removable cover to the medicinal bottle, while being appropriately positioned to allow collection of substantially all fluids from the container 10 when desired. In this example, an air-filled retractable bulb 202 is positioned proximate a fastening structure 204, such as a threading structure. When the structure 204 is fastened to the nozzle 12, the longitudinal body 206 is positioned such that the open-end tip 208 is positioned proximate the vertex of the pooling structure 14. Pressing the member 210 atop the bulb 202 causes the bulb 202 to contract and push out air from the bulb 202, assuming the bulb 202 is made of a flexible resilient material such as rubber or other elastomeric material. Pressing the member 210 causes air to escape from the open-end tip 208, consequently creating a vacuum within the body 206, upon release of the member 210 and resulting suction caused by the bulb 202 returning to it's original shape. In this manner, medicinal fluid may be drawn into the fluid retrieval device 200 while it is also serving as a cover to the medicinal container 10. Once the medicinal fluid has been drawn in, the fluid retrieval device 200 may be unthreaded from the nozzle 12, and the medicine may be administered.

FIGS. 18-19 illustrate other embodiments of a portion of a dropper 200 depicted in FIG. 17. The dropper of FIG. 18 does not include the member 210 shown in FIG. 17. Instead, the user depresses the resilient/elastomeric material 202 directly to release air from the dropper, which ultimately creates the vacuum effect upon it's release. FIG. 19 illustrates a dropper with a member 210, but illustrates that other particular shapes of the member 210 are contemplated by the invention.

Also, as previously indicated, other manners of fastening the structure 204 to the container 10 nozzle 12 are clearly within the scope of the invention. The particular fastening mechanism employed is not of particular significance to the invention. For example, while the use of threads are used for illustrative purposes, any other fastening mechanism may be used, including clamps, snaps, non-permanent adhesives, Velcro, or other fastening means.

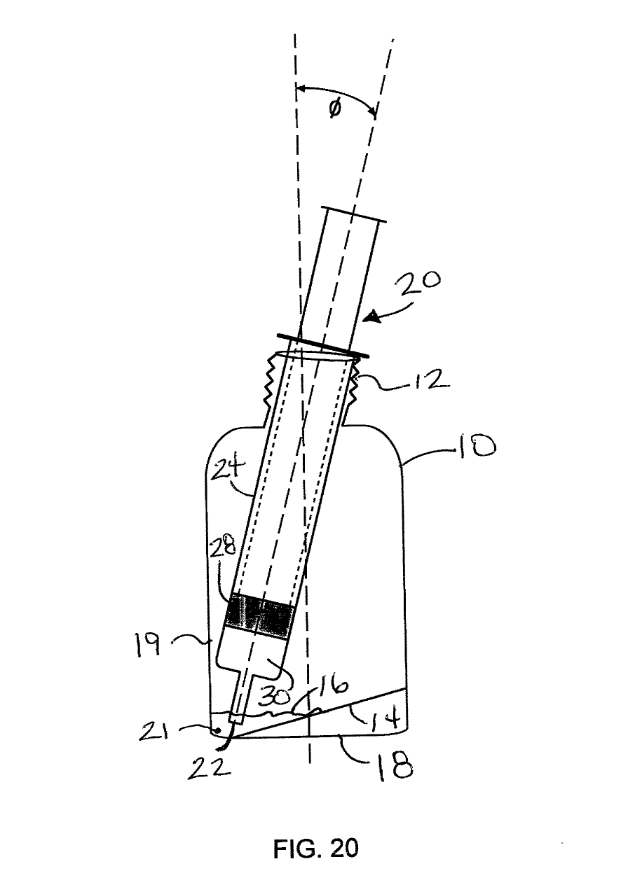

FIG. 20 illustrates another embodiment of an apparatus according to the principles of the present invention. The container 10 may be any desired shape, and in the illustrated embodiment is generally the shape of a cylinder with an opening or nozzle 12 at one end. In this example, the opening/nozzle is provided at an acute angle φ from the longitudinal axis traveling through approximately the center of the container cylinder body. The medicinal container 10 includes a fluid pooling structure 14 that facilitates the pooling of the medicinal fluid via gravitational force such that the fluid retrieval device 20 may collect the fluid. The fluid retrieval device 20, a blunt-tip syringe in the instant example, enters the medicinal container 10 through the angled nozzle 12 to reach the fluid 16. When the fluid is that a high level in the container 10, the precise depth at which the fluid retrieval device is inserted into the container is of relatively little significance. However, when the fluid 16 level is at a low level in the container 10, traditional medicinal bottles distribute the little remaining fluid over a large service area at the bottom of the medicinal container. The example embodiment of FIG. 20 utilizes a pooling structure 14 to direct the fluid to an area substantially along a longitudinal axis of the fluid retrieval device 20. In this example, the container 10 can be placed on a horizontal surface, and the fluid pooling structure 14 tends to direct the fluid to a location where the end tip 22 of the fluid retrieval device can easily access and collect substantially all of the fluid in the container 10. The fluid pooling structure 14 may include a variety of types of directional structures to direct the medicinal fluid towards the target pooling region, including a single planar surface, multiple planar surfaces, a non-uniform surface that, as a whole, is directed towards the targeted pooling region, etc.

In another embodiment of the invention, the bottom surface 18 serves as the pooling structure 14, and the container 10 is tipped at an angle such that the fluid retrieval device 20 moves through the angle φ towards a vertical position. The angled nozzle 12 allows the end tip 22 of the device 20 to be positioned proximate the structural boundary 21 between the cylindrical side wall 19 and the bottom surface 18 of the container 10. In such a case, the additional fluid pooling structure 14 is not required, and the side wall 19 and bottom surface 18 form the fluid pooling structure. However, the nozzle 12 must be angled to facilitate entry of the device 20 such that the end tip 22 may be positioned substantially proximate the structural boundary 21, and the container 10 can then be rotated such that the device 20 moves towards a vertical position to pool the fluid between the side wall 19 and bottom surface 18.

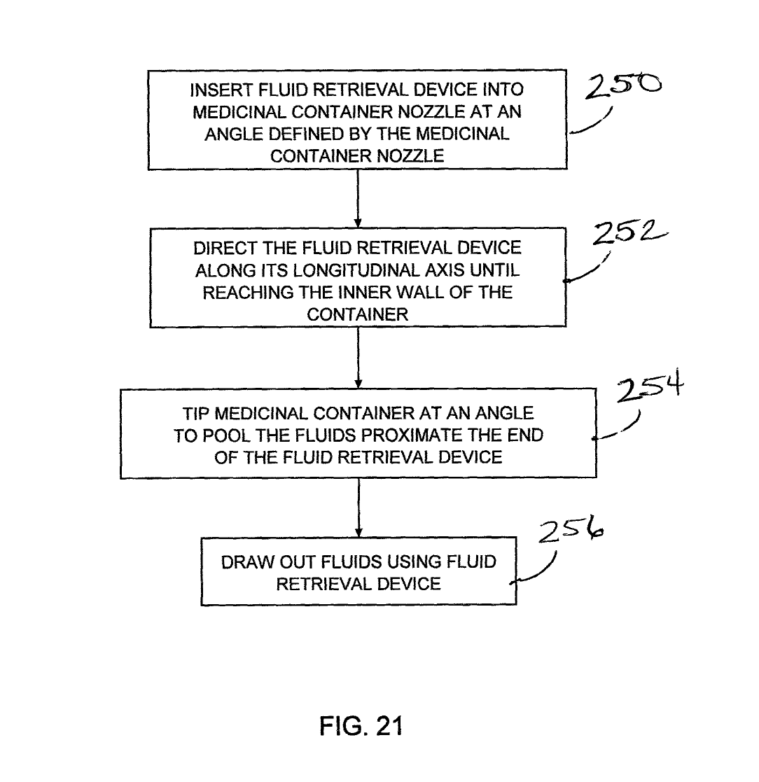

FIG. 21 is a flow diagram of a method for retrieving fluid from a container having an angled nozzle, such as the container depicted in FIG. 20. The fluid retrieval device is inserted 250 into the medicinal container nozzle at an angle defined by the medicinal container nozzle. For example, referring briefly to FIG. 20, the angle defined by the medicinal container nozzle is angle φ with respect to a vertical line. The fluid retrieval device, such as a syringe or dropper, is directed substantially along its longitudinal axis through the nozzle until it is proximate the inner wall of the container, as illustrated at block 252. Because of the angle φ , the fluid retrieval device will be substantially directed towards a “corner” of the medicinal container. The medicinal container is tipped 254 at an angle to pool the fluids proximate the tip or end of the fluid retrieval device. In one particular embodiment, the optimal angle at which the container is tipped is approximately the same as the angle φ at which the nozzle is angled with respect to the container, however the container is tipped in the reverse direction from the angle of the nozzle. For example, if the nozzle is tipped an angle φ to guide the fluid retrieval device to the “corner” of the medicinal bottle, the container is optimally tipped at an approximate angle −φ such that the fluid retrieval device tends to become more vertical. In this fashion, the fluids can be drawn out 256 using the fluid retrieval device, while pooling the fluids proximate an intersection of the bottom and side of the medicinal container.

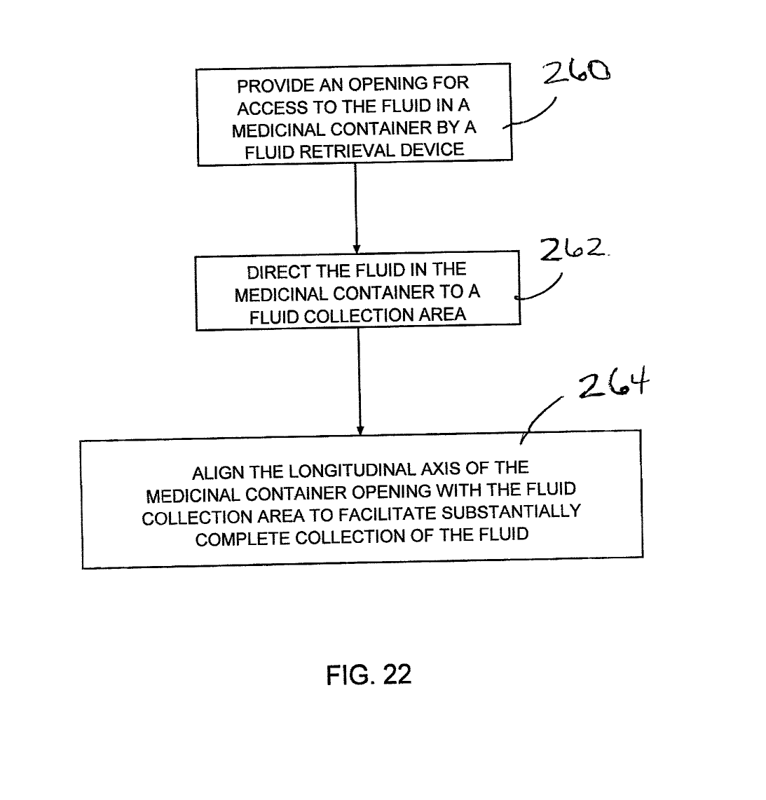

FIG. 22 is a flow diagram generally illustrating one method for maximizing the removal of fluids from a medicinal fluid container in accordance with the present invention. An opening is provided 260 for access by a fluid retrieval device to the fluid in a medicinal container. The fluid in the medicinal container is directed 262 to a fluid collection area facilitated by the structure of the medicinal container. The longitudinal axis of the medicinal container opening is aligned 264 with the fluid collection area to facilitate substantially complete collection of the fluid.

The foregoing description of the exemplary embodiment of the invention has been presented for the purposes of illustration and description. It is not intended to be exhaustive or to limit the invention to the precise form disclosed. Many modifications and variations are possible in light of the above teaching. For example, fluids other than medicinal fluids that are extracted from a container using a suction or siphon device can equally benefit from the teachings of the present invention. For example, the fluid may be a food item for a baby or toddler, such as fruit juice, rather than a medicine. Other fluids requiring administering small amounts where a dropper or syringe is useful will also benefit from the invention, such as vanilla, angostura bitters, and other cooking liquids. Fluids also encompass non-liquid substances such as powders or other substances having fluid characteristics. A wide variety of fluids may be used in connection with the apparatus and method described herein. It is therefore intended that the scope of the invention be limited not with the particular embodiments set forth in this detailed description, but rather by the claims appended hereto.

What is claimed is:

1. A medicinal container, comprising:
a container body to hold a volume of medicinal fluid;
a container nozzle coupled to the container body, the nozzle having an opening to permit passage of a fluid retrieval device into the container body; and
a fluid pooling structure tending to guide the volume of medicinal fluid towards a target pooling region in response to a gravitational force.

2. The medicinal container as in claim 1, wherein the container body includes at least one side surface and a bottom surface, and wherein the fluid pooling structure is coupled to the side surface and inclined to direct the medicinal fluid away from the side surface and towards the target pooling region.

3. The medicinal container as in claim 1, wherein the container body includes a substantially cylindrical housing, and wherein the fluid pooling structure comprises a substantially conical device coupled to the cylindrical housing to direct the medicinal fluid towards the target pooling region at a vertex of the conical device.

4. The medicinal container as in claim 1, wherein the fluid pooling structure comprises a substantially conical device coupled to the container body to direct the medicinal fluid towards the target pooling region at a vertex of the conical device.

5. The medicinal container as in claim 1, wherein the fluid pooling structure is coupled to the container body and inclined to direct the medicinal fluid away from the container body and towards the target pooling region.

6. The medicinal container as in claim 1, wherein the fluid pooling structure comprises at least one inclined surface leading to a pooling channel corresponding to the target pooling region.

7. The medicinal container as in claim 6, wherein the pooling channel is defined by a side surface and a bottom surface of the container body.

8. The medicinal container as in claim 6, wherein the fluid pooling structure comprises at least two inclined surfaces leading to the pooling channel, and wherein the pooling channel is defined by at least the two inclined surfaces.

9. The medicinal container as in claim 6, wherein the pooling channel comprises means for facilitating fluid retrieval between a fluid receiving end of the fluid retrieval device and the pooling channel.

10. The medicinal container as in claim 6, wherein the pooling channel comprises a grate.

11. The medicinal container as in claim 1, wherein the target pooling region comprises a grate.

12. The medicinal container as in claim 1, wherein the target pooling region comprises means for facilitating suction between a fluid receiving tip of the fluid retrieval device and the target pooling region when the fluid receiving tip is positioned proximate the target pooling region.

13. The medicinal container as in claim 1, wherein the container nozzle comprises threads to receive a correspondingly threaded container cap.

14. The medicinal container as in claim 1, wherein the container nozzle comprises threads to receive a correspondingly threaded component that is coupled to the fluid retrieval device, whereby the fluid retrieval device may be coupled to the container nozzle via the threads and the threaded component.

15. The medicinal container as in claim 1, wherein the fluid pooling structure forms a bottom surface of the medicinal container.

16. The medicinal container as in claim 1, wherein the fluid pooling structure comprises a false bottom in addition to a bottom surface of the medicinal container.

17. The medicinal container as in claim 1, wherein the fluid pooling structure comprises means for concentrating the volume of medicinal fluid into a physical region having a diminishing volume.

18. The medicinal container as in claim 1, wherein the fluid pooling structure comprises means for directing the medicinal fluid towards the target pooling region.

19. The medicinal container as in claim 1, wherein the fluid pooling structure comprises means for directing the medicinal fluid towards a physical region having a diminishing surface area as a fluid level of the medicinal fluid decreases.

20. The medicinal container as in claim 1, wherein the fluid pooling structure comprises means for tending to reduce the surface area of the medicinal fluid at its fluid level.

21. The medicinal container as in claim 1, wherein the fluid pooling structure comprises means for concentrating the medicinal fluid in a physical area less than that dictated by the container body.

22. The medicinal container as in claim 1, wherein the opening of the container nozzle is substantially aligned with the target pooling region to direct a fluid-receiving tip of the fluid retrieval device to the target pooling region.

23. The medicinal container as in claim 22, wherein a longitudinal axis of the container nozzle is substantially parallel to a longitudinal axis of the medicinal container.

24. The medicinal container as in claim 22, wherein a longitudinal axis of the container nozzle is offset with respect to a longitudinal axis of the medicinal container by a predetermined angle, wherein the predetermined angle facilitates directing the fluid-receiving tip to the target pooling region.

25. The medicinal container as in claim 1, wherein the container nozzle comprises means for directionally guiding the fluid retrieval device to the target pooling region.

26. A medicinal fluid dispensing system, comprising:
(a) a medicinal container, comprising:
(i) a container body to hold the medicinal fluid;
(ii) a container nozzle coupled to the container body and having an opening therein; and
(iii) a fluid pooling structure arranged to direct the medicinal fluid to a target pooling region in response to a gravitational force;
(b) a fluid retrieval device configured to pass through the opening in the nozzle; and
(c) wherein the container nozzle is configured to direct the fluid retrieval device towards the target pooling region to retrieve the medicinal fluid pooled at the target pooling region.

27. The medicinal fluid dispensing system as in claim 26, wherein the opening in the container nozzle and the fluid retrieval device are substantially the same shape such that the fluid retrieval device may be admitted into the container body via the opening.

28. The medicinal fluid dispensing system as in claim 26, wherein the opening in the container nozzle and the fluid retrieval device are substantially cylindrical.

29. The medicinal fluid dispensing system as in claim 26, wherein the fluid retrieval device is a syringe.

30. The medicinal fluid dispensing system as in claim 26, wherein the fluid retrieval device is a dropper device.

31. The medicinal fluid dispensing system as in claim 26, wherein the fluid retrieval device comprises means for drawing the medicinal fluid out of the medicinal container and into the fluid retrieval device.

32. The medicinal fluid dispensing system as in claim 26, wherein the fluid retrieval device comprises a connector for coupling to the container nozzle, thereby removably coupling the fluid retrieval device to the medicinal container.

33. The medicinal fluid dispensing system as in claim 32, wherein the connector comprises a first threaded member, and wherein the container nozzle comprises a second threaded member for mating with the first threaded member to couple the fluid retrieval device to the medicinal container.

34. The medicinal fluid dispensing system as in claim 26, wherein the fluid retrieval device comprises means for connecting the fluid retrieval device to the container nozzle.

35. The medicinal fluid dispensing system as in claim 26, wherein the configuration of the container nozzle comprises an angled nozzle such that a longitudinal axis of the container nozzle intersects with a longitudinal axis of the medicinal container at an angle that facilitates directing the fluid retrieval device towards the target pooling region.

36. The medicinal fluid dispensing system as in claim 26, wherein the target pooling region is defined by the fluid pooling structure which comprises a side wall and a bottom surface of the container body.

37. The medicinal fluid dispensing system as in claim 26, wherein the target pooling region is defined by the fluid pooling structure and a side wall of the container body.

38. The medicinal fluid dispensing system as in claim 37, wherein the fluid pooling structure comprises at least one inclined surface to direct the medicinal fluid to the target pooling region.

39. The medicinal fluid dispensing system as in claim 26, wherein the configuration of the container nozzle comprises a nozzle directed such that a longitudinal axis of the container nozzle is substantially parallel with a longitudinal axis of the medicinal container, and wherein the target pooling region is substantially centered along the longitudinal axis of the medicinal container.

40. A method for maximizing fluid extraction from a container, comprising:
providing directional access in the container for passage of a fluid retrieval device therethrough;
converging the fluid to a fluid collection area having decreased volumetric dimensions as compared to the container dimensions;
substantially aligning the longitudinal axis of the container access with the fluid collection area; and
whereby a fluid retrieval device inserted into the container via the directional access is directed to the fluid collection area as governed by the container access and aligned fluid collection area, thereby maximizing fluid extraction quantity from the container.

41. The method of claim 40, wherein converging the fluid comprises redirecting the fluid in the container to the fluid collection area.

42. A fluid reservoir system for maximizing fluid extraction therefrom, comprising:
a fluid container; means for providing directional passage by a fluid retrieval device into the container;
means for directing the fluid to a fluid collection area having decreased volumetric dimensions as compared to the container dimensions; and
means for substantially aligning the longitudinal axis of the container directional passage with the fluid collection area.

43. The fluid reservoir system as in claim 42, wherein the fluid retrieval device comprises means for retrieving the fluid from the container via the fluid collection area.

44. A method for maximizing fluid extraction from a medicinal container, comprising:
inserting a fluid retrieval device into a medicinal container nozzle substantially along a longitudinal axis of the medicinal container nozzle, wherein the longitudinal axis of the medicinal container nozzle is arranged at an acute angle and nonparallel to a longitudinal axis of the medicinal container;
directing the fluid retrieval device along a longitudinal axis of the fluid retrieval device until proximate intersecting side and bottom segments of the medicinal container;
adjusting an angle of the medicinal container to pool the fluid between the intersecting side and bottom segments proximate an end of the fluid retrieval device; and
suctioning the pooled fluid into the fluid retrieval device by way of the end of the fluid retrieval device.
